(19) United States
(12) Patent Application Publication
(10) Pub. No.: US 2018/0071145 A1
(43) Pub. Date: Mar. 15, 2018

(54) ANTI-FOG GOGGLES

(71) Applicant: Gyu Won LEE, Icheon-si (KR)

(72) Inventor: Gyu Won LEE, Icheon-si (KR)

(21) Appl. No.: 15/695,136

(22) Filed: Sep. 5, 2017

(30) Foreign Application Priority Data
Sep. 13, 2016 (KR) 10-2016-0118325

(51) Int. Cl. A61F 9/02

(52) U.S. Cl. CPC A61F 9/028

(57) ABSTRACT

Disclosed herein are anti-fog goggles. The anti-fog goggles include: a frame configured such that a lens is attached to the front surface thereof, and configured to surround the face of a wearer; at least one air outlet formed on a side of the frame, and configured to include an outlet body formed in a funnel shape whose diameter decreases backward and a filter formed on the front of the outlet body; air intakes formed between the lens and the face of the wearer; and an air transfer tube configured to connect between the air intakes and the air outlet, and configured such that a distal end thereof protrudes into the air outlet; wherein, as external air has faster flow velocity due to a difference in pressure while passing through a distal end of the air transfer tube, moisture is moved to the air outlet via the air transfer tube.

### BACKGROUND

### 1. Technical Field

The present invention relates generally to anti-fog goggles, and more specifically to anti-fog goggles which generate a difference in pressure inside the goggles based on Bernoulli's Principle and, thus, rapidly discharge moisture generated inside the goggles, thereby minimizing fog which occurs on a lens due to a difference in temperature between the inside and outside of the goggles.

### 2. Description of the Related Art

In general, goggles are used for skiing, cycling, snowboarding, motorcycling, all terrain vehicle (ATV) riding, paintballing, and military work. Goggles are commonly configured such that a head strap is connected to a frame to which protective glasses are attached, and thus a user wears the frame, to which the protective glasses are attached, on his or her face by means of the head strap.

In such conventional goggles, when there is a considerable unbalance in temperature between warm air having moisture, confined inside the goggles, and the inner surface of a goggle lens, condensation occurs, and thus fog occurs on the inner surface of the goggle lens.

In other words, when temperature and relative humidity are formed inside a space formed between the face and eyes of a user and a goggle body, a dew point is reached, and thus condensation, such as “the occurrence of fog,” occurs on the inner surface of the goggles, with the result that a front cannot be viewed via the goggles due to moisture.

Such the occurrence of fog is a problem common to the users of goggles. In order to propose a solution to the problem of the occurrence of fog inside goggles, Korean Utility Model Registration No. 0270219 discloses a method of fabricating goggles which utilize a manual air flow means in order to eliminate the occurrence of fog on a goggle lens. However, the manual air flow means still has the problem of not completely eliminating the occurrence of fog.

### SUMMARY

The present invention has been conceived to overcome the above-described problem of the conventional art, and an object of the present invention is to provide anti-fog goggles having a new structure, which can rapidly discharge vapor from the inside of goggles of a wearer by using Bernoulli's Principle, thereby minimizing the occurrence of fog attributable to a difference in temperature between the inside and outside of the goggles.

According to an aspect of the present invention, there is provided anti-fog goggles, including: a frame configured such that a lens is attached to the front surface thereof, and configured to surround the face of a wearer; at least one air outlet formed on each side of the frame; a plurality of air intakes formed in the inside of the frame; and an air transfer tube configured to connect between the air intakes and the air outlet; wherein moisture generated inside the frame is sucked via the air intakes and discharged via the air outlet.

In the anti-fog goggles, the air outlet may include an outlet body formed in a funnel shape in which the diameter of the front thereof is larger than that of the back thereof, and a filter formed on the front of the outlet body.

In the anti-fog goggles, the air outlet may be formed on both sides or one side of the frame, and may further include a protective member configured to cover the air outlet.

The anti-fog goggles may further include at least one air inlet formed through the top or bottom surface of the frame which extends between the lens and the wearer.

The anti-fog goggles may further include an accommodation part formed through the top or bottom surface of the frame which extends between the lens and the wearer and configured to accommodate the air inlet.

In the anti-fog goggles, the air inlet may include: a panel configured to be accommodated inside the accommodation part; vents configured to include a plurality of holes which penetrate the panel; and a filter configured to be accommodated inside the accommodation part, and disposed below the panel.

In the anti-fog goggles, the air transfer tube may be formed to protrude into the air outlet, and thus air entering from the outside has lower pressure while passing through the air transfer tube.

In the anti-fog goggles, the air intakes formed between the lens and the face of the wearer may be formed inside the frame.

### BRIEF DESCRIPTION OF THE DRAWINGS

The above and other objects, features and advantages of the present invention will be more clearly understood from the following detailed description taken in conjunction with the accompanying drawings, in which:

FIGS. 1 and 2 are perspective views schematically showing the appearance of anti-fog goggles according to an embodiment of the present invention;

FIG. 3 is an exploded perspective view showing a frame with a lens separated therefrom and parts of air inlets and air intakes;

FIG. 4 is a plan view showing the planar state of an anti-fog goggles according to an embodiment of the present invention; and

FIGS. 5 and 6 are views showing examples of structures in which moisture is discharged via an air outlet from the inside of anti-fog goggles according to embodiments of the present invention.

### DETAILED DESCRIPTION

Preferred embodiments of the present invention will be described in detail below with reference to the accompanying drawings.

It should be noted that the same or similar elements in the drawings are designated by the same reference symbols as far as possible even when they are shown in different drawings. Furthermore, in the following description of the present invention, when it is determined that a detailed description of a related well-known configuration or function may make the gist of the present invention obscure, the detailed description will be omitted.

Throughout the specification and the claims, when any portion is described as being “connected” to another portion, this includes both the case where the any portion is “directly connected” to the other portion and the case where the any portion is “indirectly connected” to the other portion through a third portion. Furthermore, when any portion is described as including any component, this does not mean that another component is excluded but means that the any portion may include another component unless otherwise specified.

The term “goggles” used herein refers to a structure in which a lens is coupled to the front surface of a frame forming an appearance, a cushion configured to come into contact with the face of a wearer is coupled to a side surface opposite to the surface of the frame to which the lens is coupled, and a head strap configured to surround and fasten the head part of the wearer is connected to both sides of the frame.

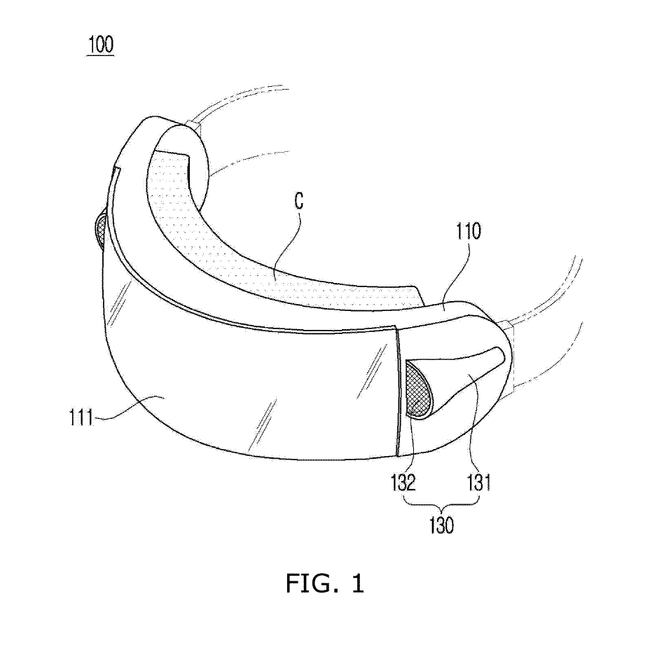

As shown in FIG. 1, anti-fog goggles 100 according to an embodiment of the present invention includes: a frame 110 configured such that a lens is attached to the front surface thereof, and configured to surround the face of a wearer; at least one air outlet 130 formed on each side of the frame 110; air intakes 140; and at least one air transfer tube 150 configured to connect between the air intakes 140 and the air outlet 130.

Referring to FIG. 1, the frame 110 forms the appearance of the anti-fog goggles 100 according to the present embodiment. The frame 110 is coupled to the lens 111 on the front surface thereof, and is formed in a shape which surrounds the eye portions of the face of a wearer. Furthermore, it is preferred that the frame 110 is formed in a curved shape so that the goggles come into tight contact with the face of a wearer and the portion of the frame 110 coming into contact with the face is made of an elastic material.

In the anti-fog goggles 100 according to an embodiment of the present invention, a cushion C may be attached to the inside of the frame 110 in order to prevent the eyes and face of a wearer from being harmed when the wearer falls on his or her face or slips.

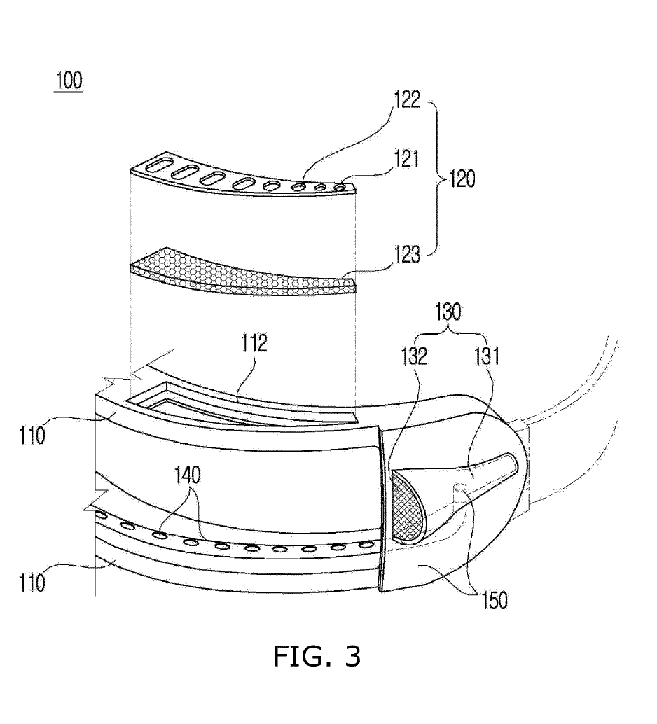

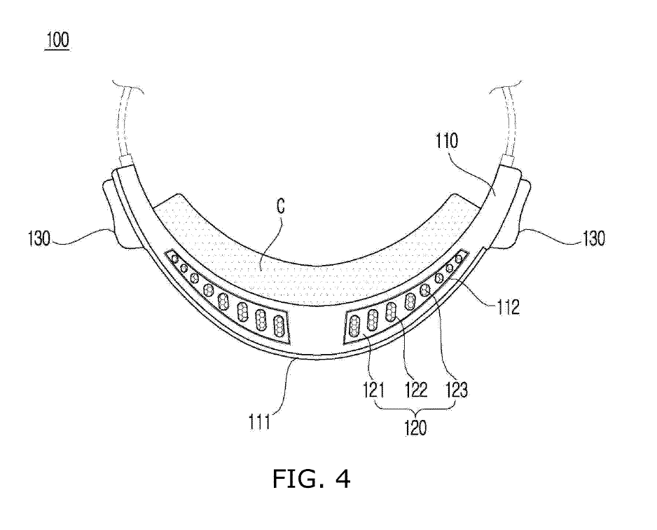

Referring to FIGS. 1 to 4, the air outlet 130 includes an outlet body 131 and a filter 132. The air outlet 130 may be disposed through each side surface of the frame 110 or through one side surface of the frame 110. The air outlet 130 functions to discharge air, entering from the outside into the goggles via the gap between the lens and the wearer or via at least one air inlet 120 located between the lens and the wearer, back to the outside. To facilitate the circulation of air inside the goggles, the outlet body 131 may be formed in a funnel shape in which the diameter of the front thereof is larger than that of the back thereof. The filter 132 is preferably formed in a grid shape and attached to the front of the body 131.

Furthermore, in the anti-fog goggles 100 according to an embodiment of the present invention, the air outlet 130 may further include a protective member (not shown) configured to cover the air outlet 130. The protective member may function to prevent the air outlet 130 from being exposed to the outside and damaged. Furthermore, the protective member may be modified in various shapes, such as a triangular shape, an elliptical shape, a rectangular shape, etc., within a range which does not hide the visual field of a wearer.

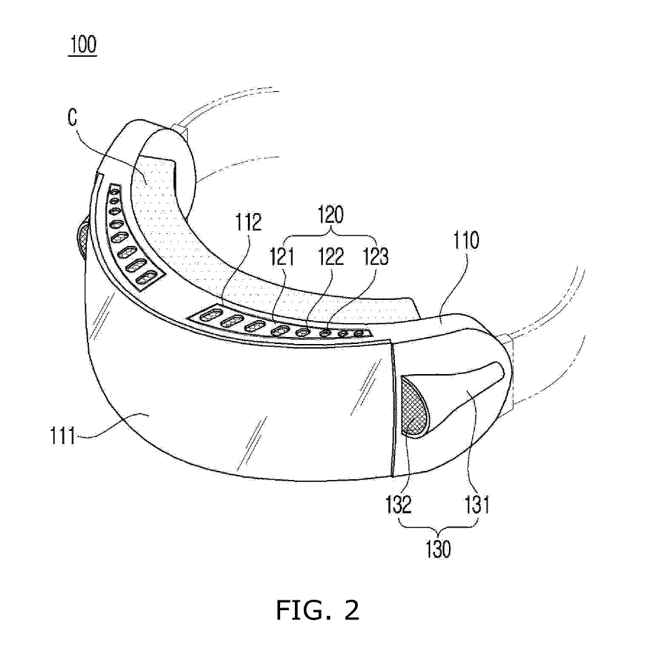

Furthermore, as shown in FIG. 2, in the anti-fog goggles 100 according to an embodiment of the present invention, the frame 110 includes at least one accommodation part 112 formed to penetrate the top surface or bottom surface of the frame 110 which extends between the front surface, to which the lens is attached, and the face of a wearer. The air inlet 120 may be accommodated inside the accommodation part.

Referring to FIG. 2, although the accommodation part 112 is formed to be located through the top surface of the frame 110 extending between the front surface, to which the lens is attached, and the face of a wearer, this is an example. The accommodation part 112 may be formed through the bottom surface of the frame 110.

The accommodation part 112 is formed through the top surface or bottom surface of the frame 110, extending between the front surface to which the lens is attached and the face of a wearer, depending on the shape of the goggles. The shape and pattern of the accommodation part 112 are not limited. As the goggles are formed to protrude from the direction of the face of a wearer, the accommodation part 112 is preferably formed to gradually become wider from both ends of the top surface of the frame 110 to the center thereof.

Furthermore, the accommodation part 112 may include a flange-shaped support extending inward so that the air inlet 120 may be accommodated inside the accommodation part 112.

The air inlet 120 is accommodated inside the accommodation part 112 which is formed through the top surface or bottom surface of the frame 110 which extends between the front surface, to which the lens is attached, and the face of a wearer the accommodation part 112. More specifically, the accommodation part 112 includes a panel 121, vents 122, and a filter 123.

In this case, the panel 121 includes the vents 122, is accommodated in the accommodation part 112, and enables air to enter from the outside of the goggles into the goggles. The vents 122 may be composed of a plurality of holes which penetrate the panel 121. Referring to FIGS. 1 and 2, the vents 122 have a shape having both arcuate ends and a predetermined length, and may be arranged to gradually become longer from one end of the panel 121 to the other end thereof. Meanwhile, the filter 123 may be disposed below the panel 121, and functions to protect the eyes of a wearer when impurities are mixed with air which enters into the goggles via the vents 122.

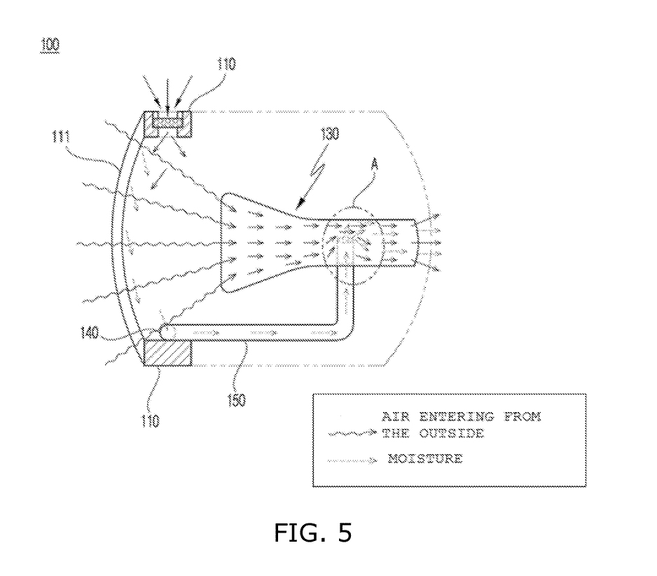

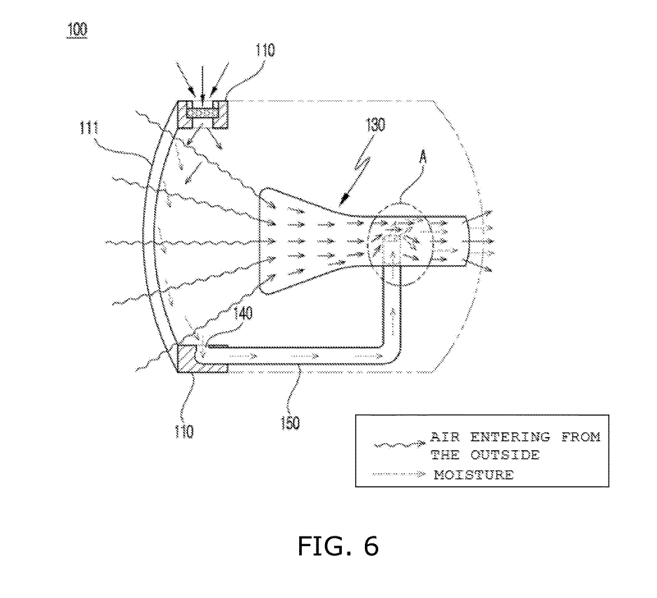

The air intakes 140 may be formed to penetrate and communicate with the inside of the frame 110, as shown in FIGS. 5 and 6, or may be exposed to the outside and formed between the lens and a wearer.

In other words, as shown in FIG. 5, the air intakes 140 may be composed of a plurality of holes which penetrate the top surface of a lower frame 110 forming the appearance of the goggles. The inside of the frame 110 is preferably formed in a tube shape so that the plurality of holes communicates with each other. Although the air intakes 140 are formed in an elliptical shape, as shown in FIG. 2, the elliptical shape may be replaced with various shapes.

Furthermore, in the case where it is difficult for the air intakes 140 to be formed to penetrate the inside of the frame, separate air intakes may be included in the lower portion of the lens, as shown in FIG. 6. Furthermore, the thickness, shape, interval, and number of holes of the air intakes 140 are not limited, and may be freely adjusted as desired.

The air transfer tube 150 may be formed such that one end thereof communicates with the air intakes 140 and the other end thereof communicates with the air outlet 130. In this case, the air transfer tube 150 is preferably formed to have the same diameter as and communicate with first ends of the air intakes 140. Furthermore, the other end of the air transfer tube 150 may protrude into and communicate with the air outlet 130.

Referring to FIGS. 5 and 6, one end of the air transfer tube 150 is formed to communicate with first ends of the air intakes 140, and the other end of the air transfer tube 150 is formed to penetrate the bottom of the air outlet 130, to pass through a virtual central point from one side of the inner circumferential surface of the air outlet 130, and to protrude to a location which is spaced apart from the opposite side of the inner circumferential surface of the air outlet 130 by a predetermined distance.

As described above, when the air transfer tube 150 is formed to protrude into the air outlet 130, air entering from the outside into the air outlet 130 has lower pressure while passing through the inside of the air outlet 130, narrowed by the air transfer tube 150, according to Bernoulli's Principle. In the anti-fog goggles 100 according to an embodiment of the present invention, as the pressure of air entering into the air outlet 130 is decreased by the air transfer tube 150 formed to protrude into the air outlet 130, suction force is generated, and thus moisture occurring on the lens 111 can be continuously discharged via the air outlet 130 by the sucking force generated due to the difference in pressure.

In other words, as shown in portions A of FIGS. 5 and 6, external air enters in front of the air outlet 130, and the entered passes through the center portion of the air outlet 130 having a smaller diameter, in which case the velocity of the entered air is increased due to the decrease in diameter and also internal pressure is decreased. Furthermore, the other end of the air transfer tube 150 is formed to extend into the air outlet, and thus the area of the air outlet 130 is further narrowed, with the result that the velocity of the entered air is further increased and also the internal pressure is further decreased according to Bernoulli's Principle. Due to the above decrease in pressure, air inside the goggles is successively sucked into the air outlet. In other words, in the anti-fog goggles 100 according to the embodiment of the present invention, moisture occurring on the lens 111 is moved to the air intakes 140 by using a difference in pressure, and moisture in the air intakes 140 is moved into the air outlet 130 having low pressure via the air transfer tube 150, thereby enabling the occurrence of moisture on the lens 111 to be minimized.

Accordingly, the anti-fog goggles according to the present invention provide the advantage of rapidly discharging moisture, generated inside the goggles due to a difference in pressure, to the air outlet via the air intakes and the air transfer tube and thus minimizing fog attributable to a difference in temperature between the inside and outside of the goggles, thereby preventing fog from occurring on the lens.

The foregoing description of the present invention is illustrative. It will be appreciated by those having ordinary knowledge in the art to which the present invention pertains that modifications to other specific shapes may be easily made without changing the technical spirit or essential features of the present invention. Therefore, it should be understood that the above-described embodiments are illustrative but not limitative in all aspects. For example, each component described in a singular form may be practiced in a distributed form, and, in the same manner, components described in a distributed form may be practiced in a combined form.

The scope of the present invention is defined by the attached claims. All variations and modifications derived from the meaning and scope of the attached claims and concepts equivalent to the claims range should be interpreted as falling within the present invention.

What is claimed is:

1. Anti-fog goggles, comprising:
a frame configured such that a lens is attached to a front surface thereof, and configured to surround a face of a wearer;
at least one air outlet formed on a side of the frame, and configured to include an outlet body formed in a funnel shape whose diameter decreases backward and a filter formed on a front of the outlet body;
air intakes formed between the lens and the face of the wearer; and
an air transfer tube configured to connect between the air intakes and the air outlet, and configured such that a distal end thereof is formed to protrude into the air outlet;
wherein as external air entering in front of the air outlet has faster flow velocity due to a difference in pressure, by which pressure in the air outlet is decreased, while passing through a distal end of the air transfer tube, moisture generated inside the lens is moved to the air outlet via the air transfer tube.

2. The anti-fog goggles of claim 1, further comprising at least one air inlet formed through a top or bottom surface of the frame which extends between the lens and the wearer.

3. The anti-fog goggles of claim 1, further comprising an accommodation part formed through a top or bottom surface of the frame which extends between the lens and the wearer.

4. The anti-fog goggles of claim 3, wherein the air inlet comprises:
a panel configured to be accommodated inside the accommodation part;
vents configured to include a plurality of holes which penetrate the panel; and
a filter configured to be accommodated inside the accommodation part, and disposed below the panel.

5. The anti-fog goggles of claim 1, wherein the air outlet further comprises a protective member.

6. The anti-fog goggles of claim 1, wherein the air intakes formed between the lens and the face of the wearer are formed inside the frame.
